(19) United States
(12) Patent Application Publication
(10) Pub. No.: US 2022/0359057 A1
(43) Pub. Date: Nov. 10, 2022

(54) Vitamin and Supplement System

(71) Applicant: Noah Myers, Albuquerque, NM (US)

(72) Inventor: Noah Myers, Albuquerque, NM (US)

(21) Appl. No.: 17/690,080

(22) Filed: Mar. 9, 2022

Related U.S. Application Data
(60) Provisional application No. 63/185,011, filed on May 6, 2021.

(51) Int. Cl. G16H 20/60

(52) U.S. Cl. CPC G16H 20/60

(57) ABSTRACT

The present invention relates generally to the field of subscription services and more, specifically to a vitamin, nutraceutical and supplement system. The system includes a delivery box or package that contains vitamins, nutraceutical and supplement bottles, and a mobile application. Through use of the mobile application, a user can create a profile for a customized vitamin, nutraceutical and supplement plan based on his or her health, wellness and fitness goals. The user then receives a regularly scheduled delivery with instructions for use.

### CROSS-REFERENCE TO RELATED APPLICATION

The present application claims priority to, and the benefit of, U.S. Provisional Application No. 63/185,011, which was filed on May 6, 2021, and is incorporated herein by reference in its entirety.

### FIELD OF THE INVENTION

The present invention relates generally to the field of subscription services. More specifically, the present invention relates to a vitamin, nutraceuticals and supplement system. The system preferably includes a delivery box that contains vitamins, nutraceuticals and supplement bottles, and a mobile application for use with creating the delivery box. Using the mobile application, users can create a customized vitamin, nutraceutical and supplement plan based on their health, fitness or other wellness goals. After the user has created the personalized plan, the individual then receives a regularly scheduled delivery via the delivery box that contains vitamins, nutraceuticals and supplements with instructions for use, including dosage and timing, as well as other warnings as may be prescribed by law, such as allergy information. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

Many individuals regularly lift weights, engage in wellness routines and exercise in an effort to keep their body and mind physically strong and healthy. Dedicated workout or fitness enthusiasts or other individuals seeking to live a healthy lifestyle often take multiple vitamins, nutraceuticals and supplements on a regular basis. The vitamins, nutraceuticals and supplements are taken to ensure a user's body is receiving the optimal amount of nutrients, along with other organic compounds that may boost athletic performance in addition to helping an individual maintain abilities of the mind to combat various mental health-related issues. However, there are a plurality of different vitamins, nutraceuticals and supplements available on the market today. Due to the general availability of a variety of products, many of which offer similar benefits, an individual must research which vitamins, supplements and nutraceuticals are right for his or her body, price-point and fitness or health-related goals. This research process could take an extremely undesirable amount of time, especially when considering the number of vitamins, supplements and nutraceuticals available for purchase and use. Further, individuals may be unaware that certain vitamins, supplements and/or nutraceuticals exist, and as a result may not know that they should be taking them in order to improve their general well-being. Further, individuals may run out of vitamins, supplements or nutraceuticals and may not have the time to travel to the store to replenish their vitamin, supplement or nutraceutical supply. In addition, the task of replenishing vitamins, supplements or nutraceuticals is often burdensome and undesirable, especially if an individual has a busy day with little free time.

Therefore, there exists a long-felt need in the art for a means to allow an individual to easily obtain vitamins, nutraceuticals and supplements. Further, there exists a long-felt need in the art for a means to quickly and easily educate an individual on a variety of vitamins, nutraceuticals and supplements. In addition, there exists a long-felt need in the art for a vitamin, nutraceutical and supplement system that provides a user with vitamins, supplements or nutraceuticals, and allow users to customize the vitamins, nutraceuticals, and/or supplements they receive from the system. Finally, there exists a long-felt need for a vitamin, nutraceutical and supplement system that does not require an individual to manually replenish his or her vitamin, nutraceutical and/or supplement supply.

The subject matter disclosed and claimed herein, in one embodiment thereof, comprises a vitamin, nutraceutical and supplement system. The system preferably includes a delivery box that contains vitamins, nutraceuticals and supplement bottles or other containers, and a downloadable mobile application. By using the mobile application, an individual can create a customized vitamin, nutraceutical, and supplement plan based on his or her health, fitness and other wellness goals. The individual then receives a regularly scheduled delivery box that contains vitamins, nutraceuticals and other supplements, instructions for use and information about each vitamin, nutraceutical or supplement.

In this manner, the vitamin, nutraceutical and supplement system of the present invention accomplishes all of the forgoing objectives and allows an individual to easily obtain vitamins, nutraceuticals and other supplements. Further, the system educates an individual on a variety of vitamins, nutraceuticals and supplements and allow the individual to customize the vitamins, nutraceuticals or supplements he or she receives. Finally, the system does not require individuals to manually replenish their vitamin, nutraceutical or supplement supply.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one embodiment thereof, comprises a vitamin, nutraceutical and supplement fulfillment system. The system allows a user to receive a regularly scheduled delivery that contains vitamins, nutraceuticals and supplements. The system uses a delivery box or other container that includes the vitamins, nutraceuticals and supplement bottles, and software that is downloaded through a mobile application on a smartphone, tablet or other suitable device. By using the mobile application, an individual can create a customized vitamin, nutraceutical and supplement plan based on his or her health, fitness or other wellness goals that are uploaded into the application. Then, a number of vitamins, nutraceuticals, and supplements are delivered to individual's home or other desired location, such as an office in a delivery box or other container with instructions for use.

The delivery box may be of any shape, and is preferably square or rectangular and is normally manufactured from a cardboard or paperboard type of material. The box is preferably of a one-piece construction, such that a lid is fixedly-attached via a fold or a hinge to at least one top edge of one side surface. Any surface of the lid may further include indicia such as but not limited to: a logo, pattern, design, symbol, advertisement, series of words, instructions, etc., that may or may not be brand, vitamin, nutraceuticals or supplement-related. The interior space of the box or container may have a plurality of dividers, slots, recesses or the like, that may be fixedly-attached to the interior surface of the box, and the dividers, slots, recesses and combinations thereof house and divide various vitamin, nutraceutical or supplement bottles, protein shakers or other various fitness-related items with the box or container. The delivery box also includes an instruction card and other instructions that may be laminated or protected via a plastic film, and that contain information such as but not limited to: a list of the contents/nutrition information of the contents of the box, the fitness, wellness and health goals of the user and instructions and recommended dosages for each supplement.

The system also includes a mobile application that allows a user to access the system. By using the application, an individual first creates a profile, and inputs information related to his or her health, fitness and wellness goals such as but not limited to: blood sugar levels, cholesterol levels, current vitamin levels, current vitamins, nutraceuticals and supplements taken, height, weight, age, gender, ethnicity, geographic area, activity level, types of sports, exercise and other activities the user partakes in, fitness goals (ex. training for a marathon), health goals (ex. raise vitamin D levels), weight loss goals, weight gain goals, weight maintenance goals, biomass index, sleep levels, energy levels, allergies/sensitivities, preferred brands, organic/Non-GMO preferences and various combination to allow an individual to create a unique profile. Alternatively, the individual may select from a pre-formatted dietary or health regime so as to avoid entering too much personal information. The mobile application then uses an algorithm to identify vitamins, nutraceuticals and/or supplements within a database that meet the goals and objectives desired, based on the profile information. The system may also provide recommendations of physicians, dieticians, nutritionists and others, such as spiritual advisors, that may be consulted as part of the algorithmic process. Further, the application has a search feature that allows a user to search for each vitamin, nutraceutical or supplement within the database. Each vitamin, nutraceutical or supplement may also have its own profile page that displays the nutritional compositions, manufacturer information, ingredients, recommended dosages, health, fitness benefits of each vitamin, nutraceutical or supplement, a photo of the product, and photo, video, text reviews of each vitamin, nutraceutical or supplement from users. The application also has a management page to allow a user to schedule the frequency of deliveries of the delivery box, and to allow the user to modify the contents and quantities of each box.

The system may also disclose a method of using the system. First, a user creates a profile and enters information related to the categories described supra. Then, the mobile application uses an algorithm to identify and suggest vitamins, nutraceuticals and/or supplements within a database that meet the goals desired based on the profile information, in addition to the recommendations of physicians, dieticians, nutritionists and other advisors that may be consulted as part of the algorithmic process. Next, the user chooses from the selection of vitamins, nutraceuticals and other supplements suggested by the system. The system then generates different options for a subscription time period for the products selected. The user may then alter the products, quantities and deliveries, based on the cost from the initial selections made by the individual. The individual is then charged a subscription fee that he or she pays via the application, in addition to selecting a delivery date for the box and location for the delivery to be made. If desired, the user may also modify his or her delivery to add, remove or change quantity sizes of the vitamins, nutraceuticals or supplements from the order before the first delivery, or after at least one delivery. Next, each vitamin, nutraceutical or supplement that was selected is placed into each compartment or area within the box. The box is then delivered to a user, and the user can take the vitamins, nutraceuticals and/or supplements according to the personalized instruction card provided, based on the information entered into the system by the individual.

Accordingly, the vitamin, nutraceutical and supplement system of the present invention is particularly advantageous as it allows a user to receive a completely customized vitamin, nutraceutical and supplement supply that is tailored to his or her specific health, wellness and fitness needs and/or goals. Further, the system provides personalized instructions and information regarding each vitamin, nutraceutical or supplement, such that a user does not have to research each vitamin, nutraceutical or supplement in-depth. Further, the customizable delivery schedule allows a user to receive the delivery box or container in a frequency that does not require a user to manually replenish his or her own vitamin, nutraceutical or supplement supply.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a delivery box of one potential embodiment of a vitamin, nutraceutical and supplement system of the present invention in a closed position in accordance with the disclosed architecture;

FIG. 2 illustrates a perspective view of one potential embodiment of a delivery box of one potential embodiment of a vitamin, nutraceutical and supplement system of the present invention in an open position in accordance with the disclosed architecture;

FIG. 3 illustrates an enhanced perspective view of one potential embodiment of a delivery box of one potential embodiment of a vitamin, nutraceutical and supplement system of the present invention in an open position with instructions in an exploded view in accordance with the disclosed architecture;

FIG. 4 illustrates a graphical view of the core components of one potential embodiment of a mobile application of one potential embodiment of a vitamin and supplement system of the present invention in accordance with the disclosed architecture; and

FIG. 5 illustrates a flow chart of a method of using one potential embodiment of a vitamin, nutraceutical and supplement system of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a means to allow an individual to easily obtain vitamins and supplements. Further, there exists a long-felt need in the art for a means to allow an individual to become educated on a variety of vitamins and supplements. In addition, there exists a long-felt need in the art for a vitamin, nutraceutical and supplement system that provides a user with vitamins, nutraceuticals and supplements, and allows a user to customize the vitamins, nutraceutical and supplements he or she receive from the system, avoiding the requirement for individuals to have to physically refill their vitamin, nutraceutical or supplement supply on their own.

The present invention, in one exemplary embodiment, is comprised of a vitamin, nutraceutical and supplement system that allows a user to receive a regularly scheduled delivery of vitamins, nutraceuticals and supplements. The system includes a delivery container such as a box that contains vitamins, nutraceuticals and supplement bottles, and a mobile application, which after downloading software via the application, allows users to create a customized vitamin, nutraceutical and supplement plan based on their health, fitness or wellness goals that are inputted into the application. After the profile information is uploaded, a number of vitamins, nutraceuticals and supplements are delivered to the home of the user in a delivery container, such as a box or package, with personalized instructions for use.

The delivery box or package may be of any geometric or fanciful shape, but is preferably square or rectangular, and is comprised of a cardboard or paperboard material. The box is preferably of a one-piece construction, such that a lid is fixedly-attached via a fold line or hinge to at least one top edge of one side surface to allow the lid to be easily closed over the contents of the box. Any surface of the lid may include indicia, such as but not limited to: a logo, pattern, design, symbol, advertisement, series of words, instructions, or combinations thereof, that may or may not relate to the brand, vitamin, nutraceutical or supplement. The interior space of the box includes a plurality of dividers, recesses or slots that may be fixedly-attached to the interior surface of the box, or removably-detachable from the box. The dividers house and divide various vitamin, nutraceuticals and supplement bottles, protein shakers or other various fitness-related items within the box or package. The delivery box may also include a personalized instruction card that may be laminated or protected via a plastic film that contains information, such as but not limited to: a list of the contents/nutrition information of the contents of the box, manufacturer information, the fitness and wellness goals of the user and instructions and recommended dosages for each supplement, vitamin and nutraceutical, as well as any warning associated with the products shipped to the individual.

The system uses a mobile application that allows an individual to access the system via software that was previously downloaded onto a smart device. A user initially creates a profile and inputs information related to his or her health, fitness and wellness, such as but not limited to: blood sugar levels, cholesterol levels, current vitamin levels, current vitamins/supplements taken, height, weight, age, gender, ethnicity, geographic area, activity level, types of sports/activities the user partakes in, fitness goals (ex. training for a marathon), health goals (ex. raise vitamin D levels), weight loss goals, weight gain goals, weight maintenance goals, biomass information, sleep levels, energy levels, allergies, sensitivities, preferred brands, organic, Non-GMO preferences and various combinations of the foregoing. Alternatively, the individual may select from a predetermined nutritional, wellness or fitness regime that may have been established by an institution, physician, athlete, celebrity or other individual. The mobile application then uses an algorithm to identify vitamins, supplements and nutraceuticals within a database that meet the goals desired, based on the profile information that an individual has uploaded to the system. The algorithm may also include the recommendations of physicians, dieticians, nutritionists, etc., that may be consulted as part of the algorithmic process. Further, the application has a search feature that allows a user to search for each vitamin, nutraceutical and supplement types or brands within the database. Each vitamin, nutraceutical or supplement may also have its own profile page that displays the nutritional compositions, recommended dosages, health, fitness and wellness benefits of each vitamin, nutraceutical or supplement, a photo of the product and photo/video/text reviews of each vitamin, nutraceutical or supplement from users. In addition, the application also has a management page to allow a user to schedule the frequency of deliveries of the box or package, and to allow the individual to modify the contents of each box, such as the quantities, size and other variables.

The present invention may also contain a method of using the system. First, a user creates a profile and enters information related to the categories described supra. Then, the mobile application uses an algorithm to identify vitamins, supplements and/or nutraceuticals contained within a database that meet the goals desired based on the profile information. An additional database may contain the recommendations of physicians, dieticians, nutritionists and other advisors that may be consulted as part of the algorithmic process. Next, the individual chooses a subscription time period from the system and is charged a subscription fee that he or she pays via the application. In addition, the individual choses the frequency of shipments when selecting his or her delivery date for the box. If desired, the user may also modify his or subscription by adding or removing vitamins, nutraceuticals or supplements from the order before the first delivery has been shipped, or after at least one delivery has been made to the recipient. Next, each vitamin, nutraceutical or supplement that was selected is placed into each compartment within the box or other package to be used for the shipment. The box is then delivered to the subscriber, and the user can take the vitamins, nutraceuticals and/or supplements according to the instruction card provided.

Accordingly, the vitamin, nutraceutical and supplement delivery system of the present invention is particularly advantageous, as it allows a user to receive a completely customized vitamin, nutraceutical and supplement supply that is tailored to his or her specific health, wellness and fitness needs and or goals. Further, the system provides instructions and information regarding each vitamin, supplement or nutraceutical, such that the subscriber does not have to research each vitamin, nutraceutical or supplement in depth prior to subscribing to the service. In addition, the customizable delivery schedule allows each subscriber to receive the delivery box in a frequency that does not require a user to have to manually replenish his or her own vitamin, nutraceutical or supplement supply.

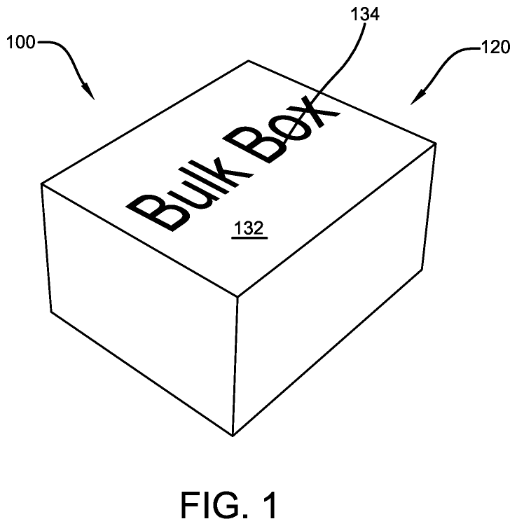

Referring initially to the drawings, FIG. 1 illustrates a perspective view of one potential embodiment of a delivery package or box 120 of one potential embodiment of a vitamin, nutraceutical and supplement system 100 of the present invention in a closed position. The system 100 includes a delivery box 120 that contains vitamins, nutraceuticals and supplement bottles 140, and a mobile application 200. Using the mobile application 200, a user can create a customized vitamin, nutraceutical and supplement plan based on his or her health, wellness and fitness goals. Then, a number of vitamins, nutraceuticals and supplements 140 will be delivered to the home in a delivery box or other package 120 with personalized instructions for use.

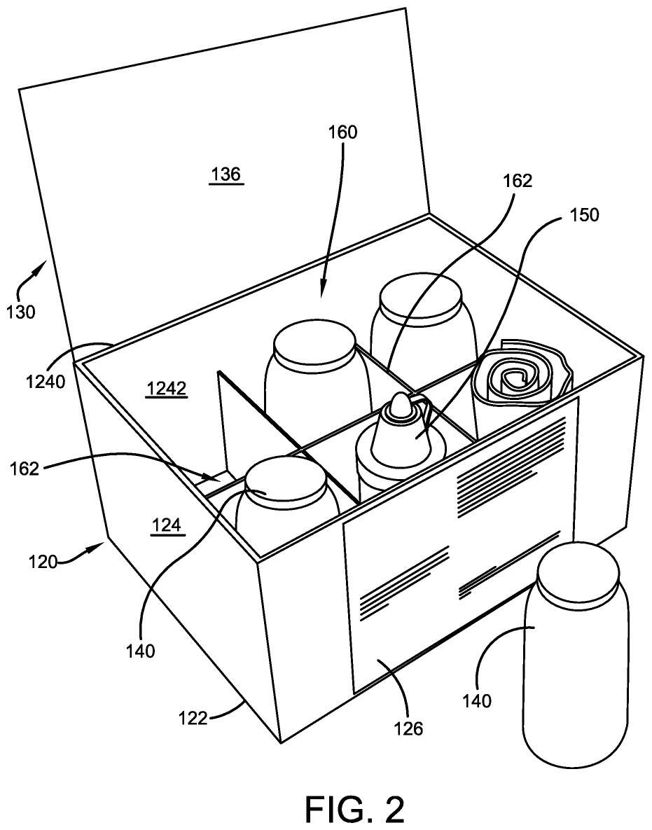

The delivery box 120 may be of any geometric shape or fanciful configuration, and in one embodiment is preferably square or rectangular, and is manufactured from a cardboard or paperboard material. The box 120 has a bottom surface 122, two sets of parallel side surfaces 124 and a top surface in the form of a lid 130. The box 120 is preferably of a one-piece construction, such that the lid 130 is fixedly-attached via a fold line to at least one top edge 1240 of one side surface 124 (see FIG. 2). Any surface of the lid 130 (but preferably the top surface 132) may further have indicia such as but not limited to: a logo, pattern, design, symbol, advertisement, series of words, instructions or combinations thereof, that may or may not be brand, vitamin or supplement-related.

FIG. 2 illustrates a perspective view of one potential embodiment of a delivery box 120 of one potential embodiment of a vitamin and supplement system 100 of the present invention in an open position. The interior space 160 of the box 120 has a plurality of dividers 162. It should be understood that rather than dividers, there may be recesses or slots inside the box to receive the products, or the products may be simply fastened to the base of the box by adhesive, ties, straps, wires or the like. The dividers 162 are preferably fixedly-attached to the interior surface 1242 of each set of side surfaces 124, such that the dividers are immovable 162. It should be appreciated that in differing embodiments, a delivery box 120 may have any number of dividers 162 that may form any number of compartments 164 that can house and divide various vitamin, nutraceuticals and supplement bottles 140, protein shakers 150 or other various fitness-related items/equipment/apparel with the box 120.

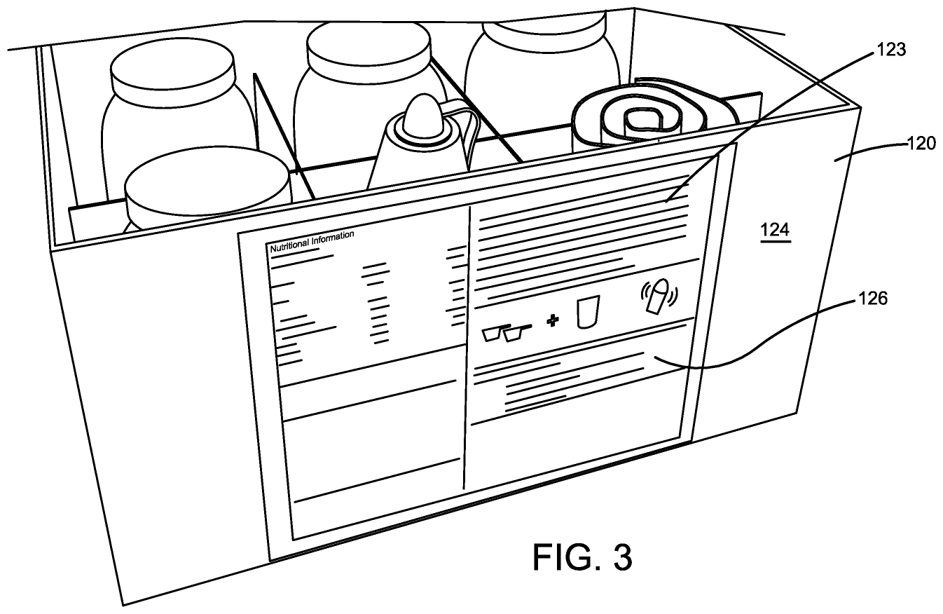

FIG. 3 illustrates an enhanced perspective view of one potential embodiment of a delivery box 120 of one potential embodiment of a vitamin and supplement system 100 of the present invention in an open position in accordance with the disclosed architecture. The delivery box 120 is also comprised of an instruction card 126 that may be laminated or protected via a plastic film. The card 126 contains information, such as but not limited to: a list of the contents and nutrition information of the contents of the box 120, the fitness, health and wellness goals of the user, instructions and recommended dosages for each vitamin, nutraceutical and supplement 140, and a small background on each vitamin, nutraceutical and supplement 140 and its uses. It is contemplated that the card 126 may be removably or fixedly-attached to any side surface 124, the top surface 132 or bottom surface 136 of the lid 130 in various embodiments, via fasteners known in the art such as adhesive, hook and loop, etc. The instruction card 126 may also include an area 123 of personalized information relating to the use of the different products provided in the box 120.

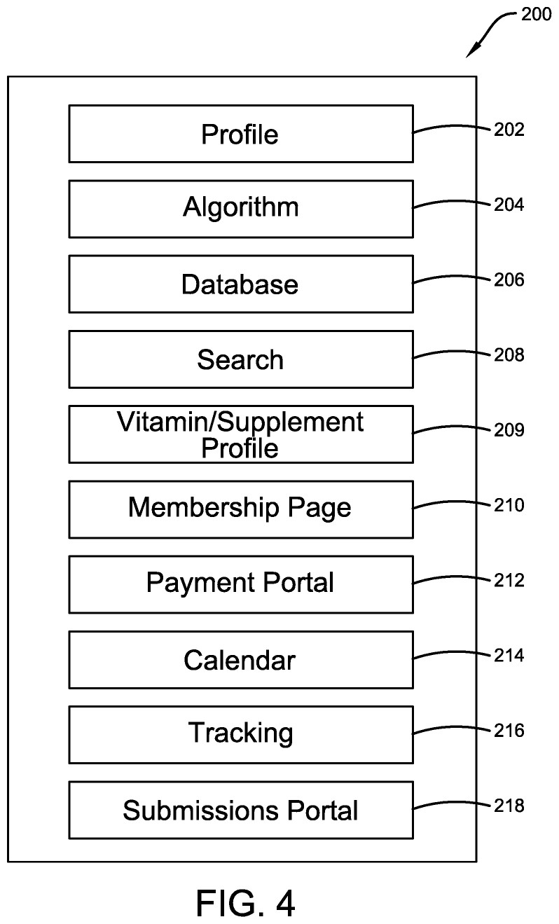

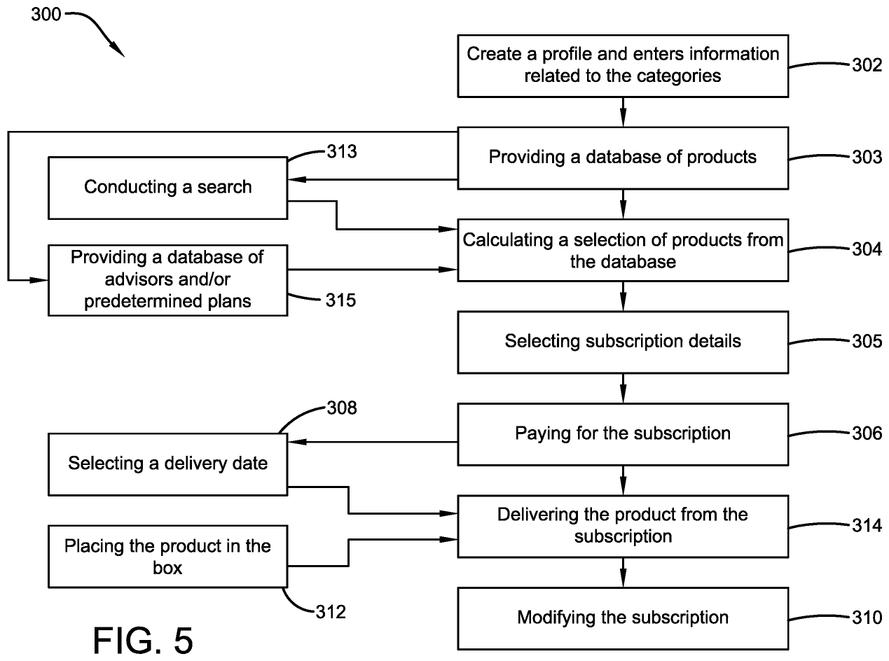

FIG. 4 illustrates a graphical view of the core components of one potential embodiment of a mobile application 200 of one potential embodiment of a vitamin, nutraceutical and supplement system 100 of the present invention in accordance with the disclosed structure. The mobile application 200 can be used on any cell phone, smart device or computer known in the art. The application 200 allows a user to access the system 100, and to enter subscription and other information. To begin using the system 100, a user first creates a profile 202 on the mobile application 200. The profile 202 allows a user to input information related to the health, fitness and wellness goals such as but not limited to: blood sugar levels, cholesterol levels, current vitamin levels, current vitamins/supplements taken, height, weight, age, gender, ethnicity, geographic area, activity level, types of sports/activities the user partakes in, fitness goals (ex. peaking for a marathon), health goals (ex. raise vitamin D levels), weight loss goals, weight gain goals, weight maintenance goals, sleep levels, energy levels, allergies/sensitivities, preferred brands, organic/Non-GMO preferences, etc. The mobile application then uses an algorithm 204 to identify vitamins, nutraceuticals, supplements within a database 206 that meet the goals desired and health, fitness and wellness requirements based on the profile 202 information, in addition to the recommendations of physicians, dieticians, nutritionists and other advisors that may be consulted as part of the algorithmic process. The advisor information may be contained in an additional database (see FIG. 5 block 315). The application 200 also has a search feature 208, that allows a user to search for each vitamin, nutraceutical or supplement within the database 206, and wherein each vitamin, nutraceutical or supplement 140 may have its own profile page 209 that displays the nutritional compositions, recommended dosages, health, fitness or wellness benefits of each vitamin, nutraceutical or supplement, a photo of the product, and photo/video/text reviews of each vitamin, supplement or nutraceutical from other users.

The application 200 also has a membership management page 210. The page 210 allows a user to choose what period of time he or she would like to be subscribed to the system 100, and receive deliveries (e.g., one month, every other month, one year, etc.). After making the selections, the user is charged a monthly or yearly subscription fee that can be paid via a payment portal 212 within the application 200 that accepts credit card, PayPal, cryptocurrency or other payment forms. The application 200 may also have a calendar 214 that a user can use to select the recurring or non-recurring date and time of each delivery of the box 120 to his or her home, residence or business, which includes intervals, such as but not limited to: every week, every other week, every month, every six weeks, every other month, every six months, every year, etc. The user may also provide variables with respect to different products so that some products may arrive more frequently than other products in the subscription service. Further, the application 200 may have a tracking feature 216 that allows a user to track the location of his or her box 120 in real-time. The membership page 210 may also allow a user to modify the subscription and to manually add or remove vitamins, nutraceutical and supplements from the order (on a per-delivery or permanent multi-delivery basis), before delivery or after at least one delivery. The application 200 may also have a submissions portal 218 that allows brands, manufacturers or companies to submit information in the form of photos, videos and text, regarding their vitamin/supplement products. The products can then be approved and uploaded to the database 206, or denied by the administrator of the application 200.

FIG. 5 illustrates a flow chart of a method 300 of using one potential embodiment of a vitamin and supplement system of the present invention in accordance with the disclosed specification. First, a user creates a profile 202 (FIG. 4) and enters information related to the categories described supra (Block 302). A database containing all of the categories shown in FIG. 4 is provided at step 303. Then, the mobile application 200 uses an algorithm 204 to identify vitamins, nutraceuticals or supplements within a database 206 that meet the goals desired based on the profile 202 information, in addition to the recommendations of physicians, dieticians, nutritionists or other advisors provided in a second database 315 that may be consulted as part of the algorithmic process (Block 304). Next, the user chooses a subscription time period at step 305 for the system 100, and is charged a subscription fee that is paid via the application 200 (Block 306). Then, a user selects the delivery date for the box 120 (Block 308). If desired, users may also modify their subscription by adding, or removing vitamins, nutraceuticals or supplements from their order before the first delivery, or after at least one delivery (Block 310). Next, each vitamin, nutraceutical or supplement 140 that was selected is placed into each compartment 164 within the box 120 (Block 312). The system may also include a search feature 313 to allow the user to select vitamins, nutraceuticals and supplements or physicians, dieticians, nutritionists and other advisors. The box 120 is the delivered to a user, wherein the user can take the vitamins/supplements 140 according to the instruction card provided 126 (Block 314).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “vitamin, nutraceutical and supplement system” and “system” are interchangeable and refer to the vitamin, nutraceutical and supplement system 100 of the present invention.

Notwithstanding the forgoing, the vitamin, nutraceutical and supplement system 100 of the present invention and its various components can be of any suitable size and configuration as is known in the art, without affecting the overall concept of the invention, provided that they accomplish the above-stated objectives. One of ordinary skill in the art will appreciate that the size, configuration and material of the vitamin, nutraceutical and supplement system 100 as shown in the FIGS. are for illustrative purposes only, and that many other sizes and shapes of the vitamin, nutraceutical and supplement system 100 are well within the scope of the present disclosure. Although the dimensions of the vitamin, nutraceutical and supplement system 100 are important design parameters for user convenience, the vitamin, nutraceutical and supplement system 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system for providing fitness and wellness products, the system comprising:
a downloadable software for use on a smart device, the downloadable software configured as an application for user interaction and information upload; and
a package configured to receive a plurality of containers having at least one of vitamins, nutraceuticals or supplements, wherein the package is comprised of a plurality of dividers, recesses or slots to receive the plurality of containers, wherein the application allows a user to select the plurality of containers for inclusion in the package.

2. The system of claim 1 further comprising at least one database containing information relating to the vitamins, nutraceuticals or supplements.

3. The system of claim 2 further comprising a second database having recommendations from at least one of a physician, a dietician, a nutritionist, and an advisor.

4. The system of claim 3, wherein the second database further comprises a selection of predetermined nutritional, wellness or fitness regimes.

5. The system of claim 1 further comprising a payment portal for accepting a payment for a system subscription.

6. The system of claim 3 further comprising a search feature to allow the user to search for at least one of vitamins, nutraceuticals or supplements, or the physician, the dietician, the nutritionist, or the advisor.

7. The system of claim 1 further comprising a tracking application for tracking the package based on the user's selection of the plurality of containers.

8. The system of claim 2, wherein the at least one database comprises information relating to a nutritional composition, a manufacturer information, an ingredient, a recommended dosage, a health and fitness benefits of the at least one of vitamins, nutraceuticals or supplements, a video, and a text review of the at least one of vitamins, nutraceuticals or supplements.

9. The system of claim 1, wherein the user interaction includes uploading an information about a subscriber to the system.

10. The system of claim 9, wherein the information includes at least one of a subscribers health, fitness and wellness goals.

11. The system of claim 10, wherein the information further comprises a height, a weight, an age, a gender, an ethnicity, a geographic area, an activity level, a weight loss goal, a weight gain goal, a weight maintenance goal, a biomass index, a sleep level, an energy level, and an allergy sensitivity.

12. A method of using a system for providing vitamins, nutraceuticals and supplements on a subscription basis, the method comprising the steps of:
downloading a software onto a smart device;
entering an information relating to a user of the smart device to create a user profile;
searching a first database having information on the vitamins, nutraceutical and supplements;
selecting at least one product from the first database;
selecting a subscription detail; and
receiving the at least one product selected from the first database.

13. The method as recited in claim 12 further comprising a step of submitting a payment for the subscription details after the step of selecting the subscription details.

14. The method as recited in claim 12 further comprising a step of modifying the subscription details after the step of selecting the subscription details.

15. The method as recited in claim 12 further comprising a step of selecting a delivery date after the step of selecting the subscription details.

16. The method as recited in claim 12 further comprising a step of searching a second database containing information from physicians, dieticians, nutritionists and other advisors.

17. The method as recited in claim 16, wherein the step of searching a second database includes searching a number of predetermined plans.

18. The method as recited in claim 12, wherein the step of creating a user profile includes a height, a weight, an age, a gender, an ethnicity, a geographic area, an activity level, a weight loss goal, a weight gain goal, a weight maintenance goal, a biomass index, a sleep level, an energy level, and an allergy sensitivity.

19. The method as recited in claim 12, wherein the first database comprises an information relating to a nutritional composition, a manufacturer information, an ingredient, a recommended dosage, a health and fitness benefit of each vitamin, nutraceutical or supplement, a video, and a text review of each vitamin, nutraceutical or supplement.

20. A system for ordering health, fitness and wellness products, the system comprising:
a software application downloaded as a mobile application on a smart device;
the mobile application requiring a user profile including one or more of height, weight, age, gender, ethnicity, geographic area, activity level, types of sports, exercise, fitness goals, health goals, weight loss goals, weight gain goals, weight maintenance goals, biomass index, sleep levels, energy levels, allergies, sensitivities, preferred brands, organic, non-GMO preferences, and various combination thereof;
a plurality of vitamins, supplements, and nutraceuticals contained in a first database;
a plurality of advisor information contained in a second database;
the mobile application allowing searching of at least the first database to select at least one of the plurality of vitamins, nutraceuticals or supplements;
the mobile application allows selection of one of the plurality of vitamins, nutraceuticals or supplements; and
the mobile application connects to a system to process the selection and arrange for delivery of the one of the plurality of vitamins, nutraceuticals or supplements.
